SEQUENCE LISTING (19 sequences)

SEQ ID NO: 1 - DNA, 5067 bp, Homo sapiens
  CDS (61)..(5049)
ctcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacctccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtcagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgccgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcctgaggaggacgaatgccaagacgaagagaaccagaaacaatgccaggacctcggcgccttcaccgagagcatggttgtctttgggtgccccaactgaccacacccccattcc

SEQ ID NO: 2 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNLRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 3 - protein, 7 aa, Homo sapiens
LPYSVVR

SEQ ID NO: 4 - protein, 6 aa, Homo sapiens
WLNEQR

SEQ ID NO: 5 - protein, 7 aa, Homo sapiens
NEQVEIR

SEQ ID NO: 6 - protein, 7 aa, Homo sapiens
AVLYNYR

SEQ ID NO: 7 - protein, 9 aa, Homo sapiens
GYTQQLAFR

SEQ ID NO: 8 - protein, 10 aa, Homo sapiens
DFDFVPPVVR

SEQ ID NO: 9 - protein, 10 aa, Homo sapiens
IHWESASLLR

SEQ ID NO: 10 - protein, 12 aa, Homo sapiens
VSHSEDDCLAFK

SEQ ID NO: 11 - protein, 13 aa, Homo sapiens
AAVYHHFISDGVR

SEQ ID NO: 12 - protein, 14 aa, Homo sapiens
AGDFLEANYMNLQR

SEQ ID NO: 13 - protein, 16 aa, Homo sapiens
SYTVAIAGYALAQMGR

SEQ ID NO: 14 - protein, 16 aa, Homo sapiens
VHQYFNVELIQPGAVK

SEQ ID NO: 15 - protein, 19 aa, Homo sapiens
DAPDHQELNLDVSLQLPSR

SEQ ID NO: 16 - DNA, 20 bp, Artificial Sequence
  Description of Artificial Sequence Primer
ggtcaagcaggactccttgt

SEQ ID NO: 17 - DNA, 21 bp, Artificial Sequence
  Description of Artificial Sequence Primer
cccttgttcatgatgaggtag

SEQ ID NO: 18 - DNA, 20 bp, Artificial Sequence
  Description of Artificial Sequence Primer
accacagtccatgccatcac

SEQ ID NO: 19 - DNA, 20 bp, Artificial Sequence
  Description of Artificial Sequence Primer
tccaccaccctgttgctgta

(19) United States
(12) Patent Application Publication
(10) Pub. No.: US 2005/0055735 A1
(43) Pub. Date: Mar. 10, 2005

(54) Use of complement protein C3 and its derivatives in enhancing mammalian embryo development

(76) Inventors: William Shu-Biu Yeung, Hong Kong (CN); Calvin Kai-Fai Lee, Hong Kong (CN); John Moon-Ching Luk, Hong Kong (CN); Cherie Yin-Lau Lee, Hong Kong (CN)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/928,312

(22) Filed: Aug. 30, 2004

Related U.S. Application Data
(60) Provisional application No. 60/501,127, filed on Sep. 8, 2003.

(51) Int. Cl. A01K 67/027

(52) U.S. Cl. 800/21

(57) ABSTRACT

The present invention relates to complement proteins, in particular, C3 protein, and methods using the same for enhancing the development of preimplantation mammalian embryos in vitro for use in assisted reproductive technologies. In particular, the present invention relates to supplementation of complement C3 protein, its precursors, fragments, or derivatives, to culture media to improve the development of cultured embryos and, thereby, enhance pregnancy rates of in vitro fertilization.

This application claims priority benefit to U.S. provisional application No. 60/501,127 filed Sep. 8, 2003, which is incorporated herein by reference in its entirety.

### 1. FIELD OF THE INVENTION

The present invention relates to complement proteins and methods of use thereof for enhancing the culturing or development of cells, tissues and embryos. In a preferred embodiment, the present invention relates to the culturing or development of mammalian embryos in vitro for use in assisted reproductive technologies. The present invention also relates to supplementation of complement proteins, in particular, C3 and its fragments, precursors, derivatives, analogs, and variants thereof to culture media to improve the development of cultured embryos and cells derived from embryos, thereby, enhance pregnancy rates of in vitro fertilization.

### 2. BACKGROUND OF THE INVENTION

In vitro fertilization and transfer of the resultant embryo to a receptive uterus is an acceptable way to overcome infertility from a variety of causes. About 15% of women of childbearing age in the United States have received an infertility service. In 2001, Centers for Disease Control and Prevention (CDC) estimated that about 1% of the total births in the United States were the result of assisted reproduction treatment (ART) (CDC, American Society for Reproductive Medicine, Society for Assisted Reproductive Technology, and RESOLVE. 2003, 2001 Assisted reproductive technology success rates, National summary and fertility clinic reports). The total number of reported ART procedures increased 66%, from 64,724 in 1996 to 107,587 in 2001 (CDC 2003, Assisted Reproductive Technology Surveillance—United States, 2001,). As a result of replacement of several embryos to the patients, multiple births (54% in 2001) were substantially higher than the overall national average of 3% (CDC 2003, Assisted Reproductive Technology Surveillance—United States, 2001,). Multiple births are a major factor in the costs attributable to assisted reproduction treatment (Katz et al., 2002, The economic impact of the assisted reproductive technologies, Nat. Cell Biol., 2000 Suppl:S29-S32). The method commonly used to reduce multiple pregnancy is to reduce the number of embryo replaced. The transfer of an embryo at the blastocyst stage allows selection of the best quality embryo for replacement without compromising the success rate due to the transfer of fewer embryos. Coculture (the culture of embryo with somatic cells) and sequential culture (the culture of embryo at different stages of development with different culture media) are the most common methods for enhancing the development of embryo to blastocyst in culture.

Since the birth of the first in vitro fertilization-embryo transfer (IVF/ET) baby, research has been ongoing to optimize the embryo culture condition in order to enhance the success rate of this expensive treatment. In general, the steps in in vitro fertilization and embryo transfer involve induction of ovulation, retrieval of oocytes, fertilization in vitro, and transfer of the embryo to a receptive uterus. The period during which embryos are cultured in vitro after fertilization is equivalent to the period during which they should be developing in the oviduct in vivo. It is generally accepted that the oviductal microenvironment provides the best support to early embryo development. Oviductal cell coculture, the culture of embryos with oviductal cells, has been shown to improve the success rate in prospective randomized control clinical trials (Yeung et al., 1996, J. Assist. Reprod. Genet., 13:762-767; see also Yeung et al., 2002, Reprod. Med. Rev., 10:21-44). Human oviductal cell coculture enhances the hatching rate and reduces the fragmentation rate of human embryos (Bongso et al., 1989, Hum. Reprod., 4:706-713; Yeung et al., 1992, Hum. Reprod., 7:1144-1149), increases the blastulation rate and total cell count per blastocyst of mouse embryos (Liu et al., 1995, Hum. Reprod., 10:2781-2786).

Despite the success of coculture, it has not been the main method for embryo culture in IVF/ET programs because of the complexity in its implementation as a routine service. Therefore, the recent development of sequential culture, i.e., the use of different culture media for culturing embryos at different stages of development, has rapidly become the method of choice for improving the outcome of human IVF (Gardner et al., 1998, Hum. Reprod., 13:3434-3440). However, the sequential culture system is not yet optimal as the development of human embryos in sequential culture system can be improved by supplementing granulocyte-macrophage colony-stimulating factor (Sjoblom et al., 1999, Hum. Reprod., 14:3069-3076), a cytokine with peak expression during the preimplantation period in the human fallopian tube (Zhao and Chegini, 1994, J. Clin. Endocr. Metab., 79:662-665). Mouse blastocysts after the coculture with human oviductal cells in G1.2 and G2.2, the most commonly used commercial sequential culture media, have better trophectoderm development and therefore, hatch more often than those cultured in sequential media alone (Xu J S et al., 2004 Mol. Reprod. Dev., 68:72-80). Thus, the beneficial effect of coculture and sequential culture on embryo development can be merged if the embryotrophic factors from cocultured cells are known and are supplemented to the sequential culture system.

In a coculture system, oviductal cells improve embryo development via various mechanisms, including the suppression of apoptosis (Xu et al., 2000, Fertil. Steril., 74:1215-1219) and caspase activity, and maintenance of mitochondrial function. (Xu et al, 2003, Fertil. Steril., 80:178-183). Oviductal cells also influence the expression of a number of genes in the early embryo, including eIF-1A, ezrin and NHE (Lee et al., 2001, Biol. Reprod., 64:910-917; Lee et al., 2003, Biol. Reprod., 68:375-382). The former two genes are associated with gene activation and development of embryos (Davis et al., 1996, Dev. Biol., 174:190-201; Louvet et al., 1996, Dev. Biol., 177:568-579) while NHE is a Na+/H+ exchanger located in the trophectoderm of blastocysts (Barr et al., 1998, Mol. Reprod. Dev., 50:146-153). On the other hand, preimplantation embryos also affect the gene expression of the oviduct (Lee et al., 2002, Biochem. Biophys. Res. Commun., 292:564-570). These observations indicate the existence of communication between the embryo and the oviduct.

Various data suggest that human oviductal cells improve the development of mouse embryo in vitro by the production of growth factors, cytokines (e.g., Yeung et al., 1996, J. Assist. Reprod. Genet., 13:772-775) and other novel factors. The identification of the oviduct derived embryotrophic factors has been difficult due to the minute amount of these factors produced by the cells. In the present invention, we have identified complement protein C3 as an embryotrophic factor produced by the human oviductal cells. The supplementation of complement protein C3 and its derivatives to the present culture system would improve the development of cultured blastocysts and enhance the pregnancy rate.

### 3. SUMMARY OF THE INVENTION

The present invention is based, in part, on the inventors' discovery that culture media when supplemented with complement C3, its precursors and/or derivatives and used for the development of embryos, in vitro improves the percentage of hatched embryos used for implantation. The present invention is particularly useful in improving the pregnancy rate in mammals.

The invention relates to in vitro culture, e.g., proliferation, growth and/or maintenance of embryos, and cells derived from embryo, said cells include, cells from preimplantation embryos at all stages of development, such embryos include, for example, but not limited to, morulas, blastocysts or cells of at least 2-4, 4-8, 8-16, 16-32, 32-64, 64-128 cells.

The present invention relates to methods and compositions for enhanced development and culturing of embryos, and cells derived from embryo, in vitro by supplementation of culture media with complement C3, its fragments, precursors and/or derivatives, analogs and variants thereof. In a preferred embodiment, the methods and compositions of the invention are useful for the enhanced development of preimplantation embryos. The invention may improve the quality of the cultured embryo, and allow the transfer of fewer good quality embryos while maintaining a high pregnancy rate, thereby, reducing the number of multiple births.

In one aspect, the present invention provides a method for developing a preimplantation mammalian embryo in vitro comprising culturing a mammalian embryo in a medium comprising a purified complement protein, precursor thereof or derivative thereof, and developing the embryo to the blastocyst stage. In certain embodiments, the purified complement protein is C3 or fragment, precursor, derivative, analog or variant thereof. In preferred embodiments, the complement protein is selected from the group consisting of ETF-3, C3, C3i, C3a, C3b and iC3b and their fragments, including C3c, C3d, C3dg, C3g, C3e and C3f, derivatives, analogs, and variants thereof. In certain embodiments, the complement protein is present at a concentration of 0.01 μg/ml to 1000 μg/ml, in other specific embodiments, the complement protein is present at a concentration of 0.1 μg/ml to 100 μg/ml, preferably 0.1 μg/ml to 10 μg/ml, more preferably, 1 μg/ml to 10 μg/ml. In specific embodiments, the concentration of complement C3 in serum is about 1500 μg/ml. In other embodiments, the complement protein is present at a physiological concentration that enhances the development of mammalian embryos as compared to the development of embryos cultured in a medium without a complement protein.

In another aspect, the present invention provides methods and compositions to improve the cell function of pre-implantation embryos, and cells derived from these embryos. Such functions include differentiation, cell proliferation, production of protein, gene expression, production of growth factor, and modulation of cell membrane permeability.

In another aspect, the present invention provides methods and compositions to improve embryo development by enhancing the hatching rate from the zona pellucida, increasing the blastulation rate and total cell count per blastocyst of an embryo, promoting cavitation, and blastulation, etc.

In yet another aspect, the present invention provides a composition comprising a culture medium comprising a complement protein or its fragments, precursors, derivatives, analogs and variants thereof. In certain embodiments, the purified complement protein is C3 or its fragments, precursors, derivatives, analogs and variants thereof. In preferred embodiments, the complement protein is selected from the group consisting of ETF-3, C3, C3i, C3a, C3b and iC3b and their fragments, such as C3c, C3d, C3dg, C3g, C3e and C3f, derivatives, analogs and variants thereof. In certain embodiments, the complement protein is present at a concentration of 0.01 μg/ml to 1000 μg/ml, in other specific embodiments, the complement protein is present at a concentration of 0.1 μg/ml to 100 μg/ml, preferably 0.1 μg/ml to 10 μg/ml, more preferably, 1 μg/ml to 10 μg/ml. In another specific embodiment, the concentration of complement C3 in serum is about 1500 μg/ml. In other embodiments, the complement protein is present at a physiological concentration that enhances the development of mammalian embryos as compared to the development of embryos cultured in a medium without a complement protein.

3.1. Definitions

As used herein, the term “embryo” refers to an animal in early stages of growth following fertilization up to the blastocyst stage. An embryo is characterized by having totipotent cells, which are undifferentiated. A blastocyst hatches from the zona pellucida and implantation commences. The term “embryo” also includes an embryo derived by nuclear transfer technique, i.e., the fusion of a nucleus of a cell with an enucleated oocyte, and by blastomere reconstruction, i.e., placing blastomere of different embryos together.

As used herein, the term “preimplantation embryo” refers to an embryo not yet implanted in the uterus to begin a pregnancy. Preimplantation embryos include, but are not limited to, embryos having at least 2-4 cells, 4-8 cells, 8-16 cells, 16-32 cells, 32-64 cells, 64-128 cells, including morulas and blastocysts.

As used herein, the term “cells derived from an embryo” includes one or more cells removed from an embryo to develop into a separate embryo.

As used herein, the term “complement” refers to a protein of the complement activation pathway. Complement proteins include, but are not limited to C1 (C1q, C1r, C1s), C2 (C2a and C2b), C3 (C3i, C3a, C3b, iC3b, C3c, C3d, C3dg, C3g, C3e and C3f) and C4 (C4a, C4b and iC4b). A complement protein can be from any species, including bovine, ovine, porcine, equine, avian, and preferably human, in native-sequence, fragment, precursor, analog, derivative, variant or complex form, and from any source, whether natural, synthetic, or recombinant.

As used herein the terms “polypeptide” and “protein” refer to a polymer of amino acids of three or more amino acids in a serial array, linked through peptide bonds. The term “polypeptide” includes proteins, protein fragments, protein analogues, oligopeptides and the like. The term “polypeptides” contemplates polypeptides as defined above that are encoded by nucleic acids, produced through recombinant technology, isolated from an appropriate source such as a mammal, or are synthesized. The term “polypeptides” further contemplates polypeptides as defined above that include chemically modified amino acids or amino acids covalently or noncovalently linked to labeling ligands.

As used herein, the term “precursor” refers to a proteinaceous substance, from which another, usually more active or mature substance, is formed, or from which a protein sequence is derived.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an protein may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived. Derivative also encompasses fragments of full-length proteins that are, for example, 5, 10, 15, 20, 25, 30, 35, 40, 45, 50, 55, 60, 65, 70, 75, 80, 85, 90, 95, 100, or multiples thereof, or more contiguous amino acids of the full-length protein.

As used herein, the term “analog” in the context of a proteinaceous agent (e.g., proteins, polypeptides, peptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA, 87:2264-2268, modified as in Karlin and Altschul, 1993, Proc. Natl. Acad. Sci. USA, 90:5873-5877. Such an algorithm is incorporated into the NBLAST and XBLAST programs of Altschul et al., 1990, J. Mol. Biol., 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucl. Acids Res., 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “purified” refers to achieving at least one order of magnitude of purification, preferably two or three orders of magnitude, most preferably four or five orders of magnitude of purification of the starting material or of the natural material. Thus, the term “purified” as used herein does not mean that the material is 100% purified and thus does not mean that a purified protein or a nucleic acid excludes any other material. In specific embodiments, a purified ARP is at least 60%, at least 80%, or at least 90% of total protein or nucleic acid, as the case may be, by weight. In a specific embodiment, a purified protein is purified to homogeneity as assayed by, e.g., sodium dodecyl sulfate polyacrylamide gel electrophoresis, or agarose gel electrophoresis.

As used herein, the term “isolated” in the context of a proteinaceous agent (e.g., a peptide, polypeptide, fusion protein, or antibody) refers to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous protein, polypeptide, peptide, or antibody (also referred to as a “contaminating protein”). The peptides of the present invention can be purified to homogeneity or any other degrees of purity. When the proteinaceous agent is recombinantly produced, it is also preferably substantially free of culture medium, i.e., culture medium represents less than about 20%, 10%, or 5% of the volume of the proteinaceous agent preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90% 95% or 98%) identical to each other typically remain hybridized to each other. Such stringent conditions are known to those skilled in the art and can be found in Ausubel et al. CURRENT PROTOCOLS IN MOLECULAR BIOLOGY (John Wiley & Sons, N.Y., 1988). In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids that hybridize under stringent conditions do not include nucleic acid molecules that hybridize under these conditions solely to a nucleotide sequence consisting of only A or T nucleotides.

As used herein, “improved function” of an embryo refers to improved potential for normal development and offspring production. This potential for embryos is assessed by evaluating cell numbers, rate of formation, size and hatching of blastocyst. “Improved function” means that the embryo has enhanced performance as assessed by one of these assays when treated with a complement, in particular, C3 protein molecule, or its precursors or derivatives under conditions described herein as compared to a control (i.e., no treatment with a complement protein).

### 4. BRIEF DESCRIPTION OF THE FIGURES

The following figures illustrate the embodiments of the invention and are not meant to limit the scope of the invention encompassed by the claims.

FIG. 1: Nucleotide (SEQ ID NO: 1) and amino acid (SEQ ID NO:2) sequences of Human Complement C3 (Genbank Accession No. NM_{—}000064). The sequences are identical to those of embryotrophic factor, ETF-3, from human oviductal cells shown in Table 1.

FIG. 2: Effects of monoclonal anti-ETF-3 antibody on the embryotrophic activity of ETF-3.

FIG. 3: SDS-PAGE and western blot analysis of protein-G purified ETF-3. Immunoblot using purified clone 14 antibody showed a band of 115 kDa (PG-115) in immunocomplex (lane A) whereas no such band was found in purified antibody alone (lane B).

FIG. 4: Two-dimensional PAGE protein profile of protein-G purified ETF-3. The protein was resolved by 2D-PAGE using 3-10 pI value and silver stained. The 115 kDa protein (arrow) reacted with the monoclonal anti-ETF-3 antibody in previous western blot analysis.

FIG. 5: Peptide mass fingerprint obtained for the 115 kDa protein. The protein was separated by SDS-PAGE and visualized by Coomassie Blue staining. The protein was subjected to in-gel tryptic digestion and analysed by MALDI-TOF MS.

FIG. 6: SDS-PAGE and western blot analysis of complement proteins C3 (lane 1), C3b (lane 2), iC3b (lane 3), purified (lane a) and partially purified ETF-3 (lane b) using monoclonal anti-ETF-3 antibody (B and E) and anti-C3 polyclonal antiserum (C and F). Immunoblot using anti-C3 antibody showed bands of α-115 and β-75 in C3 (lane C1), α′-106 and β-75 in C3b (lane C2) and β-75, α′-63 and α-40 in iC3b (lane C3) whereas only α-115, α′-106 and α-40 were found using anti-ETF-3 antibody in C3 (lane B1), C3b (lane B2) and iC3b (lane B3) respectively. Bands of 190, 115, 106, 75 and 40 were obtained in both purified (lane Fa) and partially purified ETF-3 (lane Fb) using anti-C3 whereas only bands of 115, 106 and 40 were obtained using purified clone 14 antibody (E). A and D show the Coomassie blue staining of the two sets of protein respectively.

FIGS. 7A-7I: Immunohistochemical staining of complement C3 in paraffin section of human oviduct under confocal microscope (A-I, 630×) using anti-C3 polyclonal antibody. (A), (D), (G) are bright field images; (B), (E), (H) are fluorescent images with C3 stained with FITC; (C), (F), (I), fluoresecent images showing nuclei stained with propidium iodide. C3 immunoreactivity is localized to the epithelial lining of the human oviduct (B). The signal for C3 immunoreactivity is absent after the antibody is preabsorbed with ETF-3 (E) and iC3b (H).

FIG. 8A-8D: Immunohistochemical staining of complement C3 in immortalized human oviductal cells, OE-E6/E7 using anti-C3 antibody. The cells are stained positively with anti-C3 antibody (B). The signal is absent after preabsorption of anti-C3 antibody with ETF-3 (D). (A) and (C) are phase contrast images of cultured OE-E6/E7.

FIGS. 9A-9E: Immunohistochemical staining of complement C3 in paraffin section of mouse oviduct (A-F, 200×) using anti-C3 polyclonal antibody. (A), (D) are bright field images; (B) is a fluorescent image of C3 stained with FITC; (C), (F) are fluoresecent images showing nuclei stained with propidium iodide. C3 immunoreactivity is localized to the epithelial lining of the mouse oviduct (B). No signal was obtained when anti-C3 antibody was omitted (E).

FIG. 10: Detection of human C3 in different cells and tissues by RT-PCR. Products of size 972 bp were obtained in cultured primary OE cell from patient number 109 (lane 1), 89 (lane 2), immortalized OE-E6/E7 at passages 14 (lane 3), 25 (lane 4), human oviductal epithelium tissue from 2 different patients (lane 5 and 6), SKOV-3 cell (lane 7) and human liver tissue (lane 9). No C3 was found in CHO-K1 cell (lane 8) and dH₂O control (lane 10). M: One-kb DNA marker. GAPDH: Glyceraldehyde-3-phosphate dehydrogenase (amplification control).

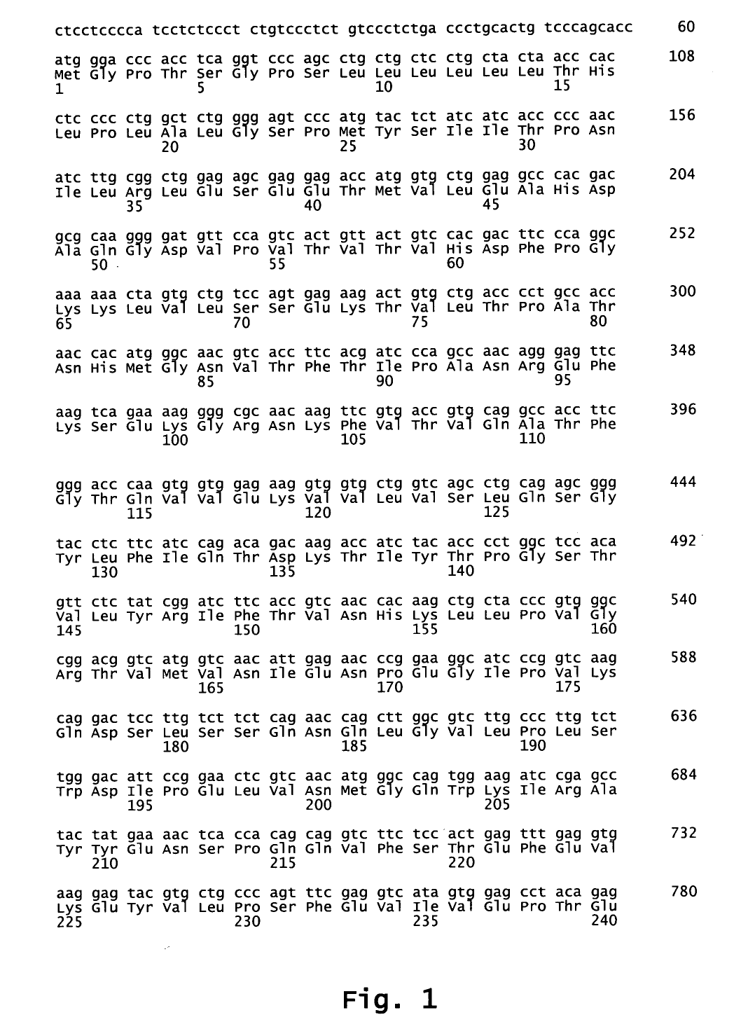

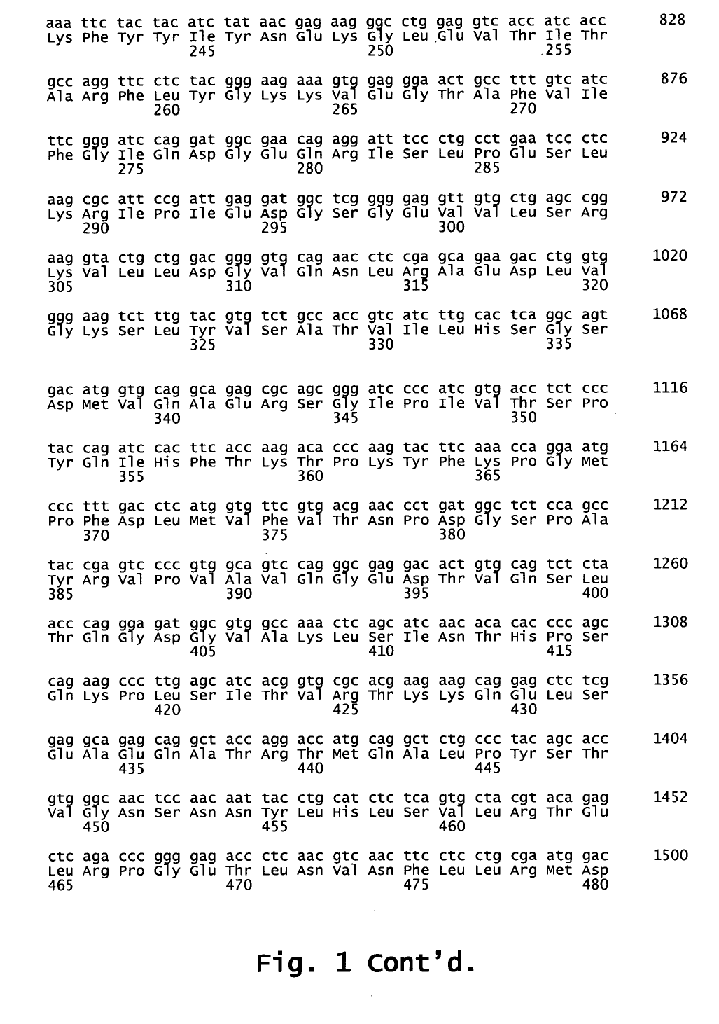

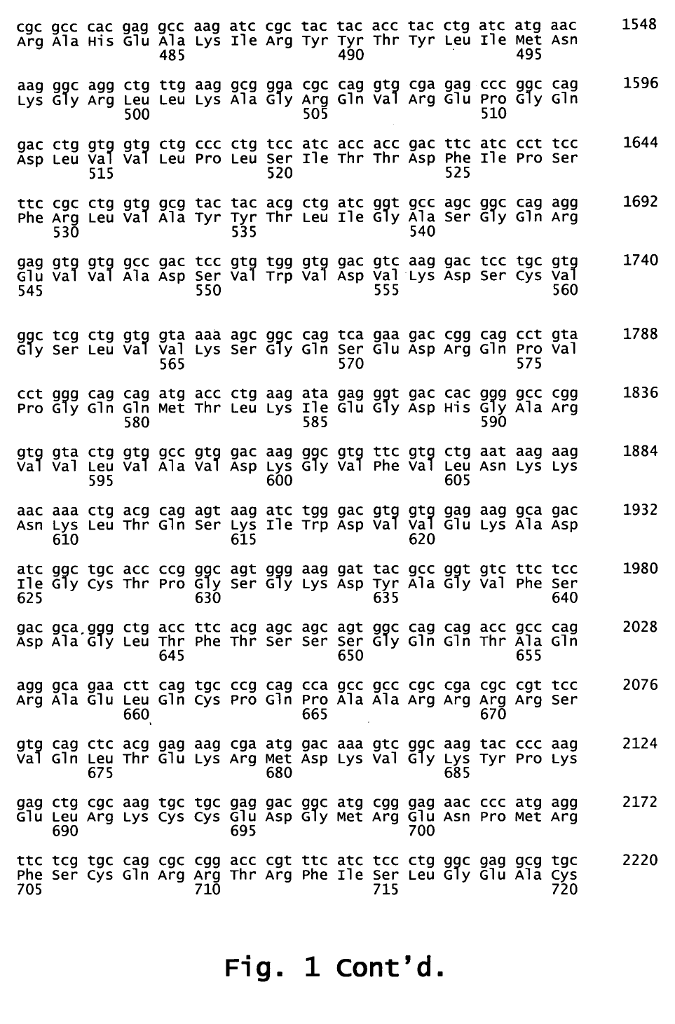

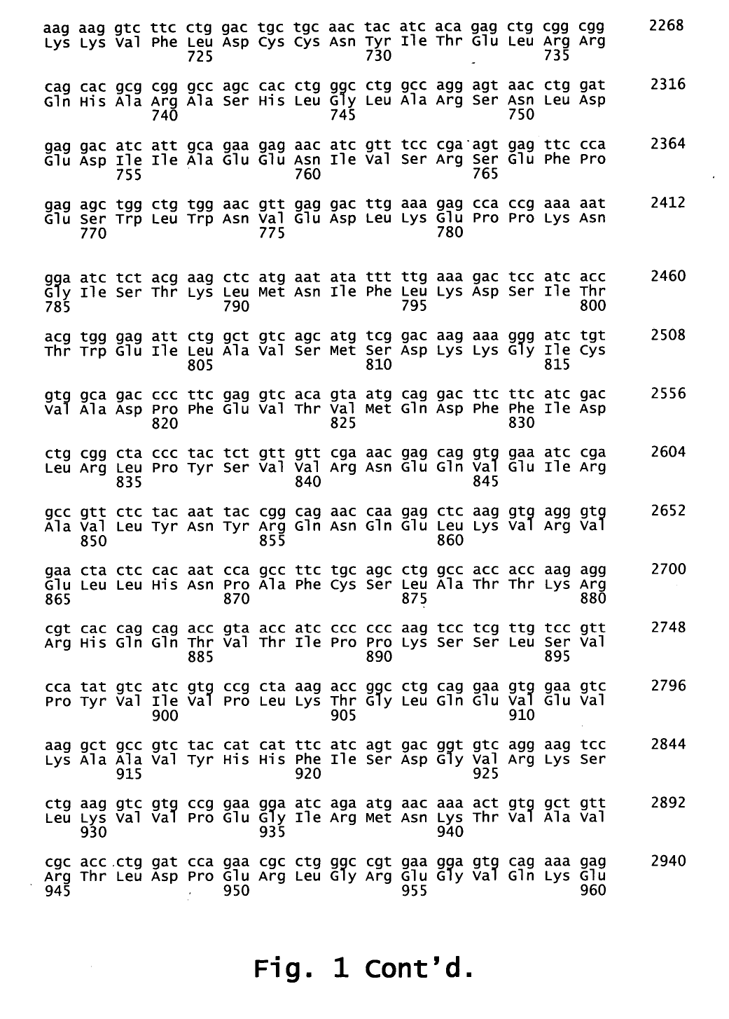

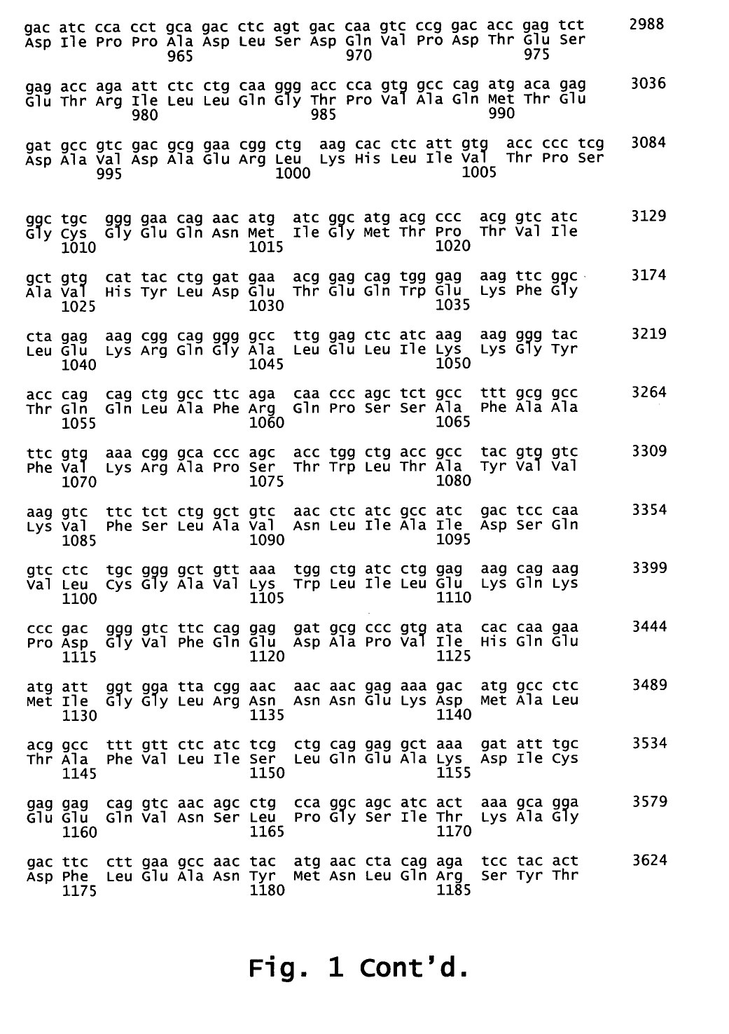

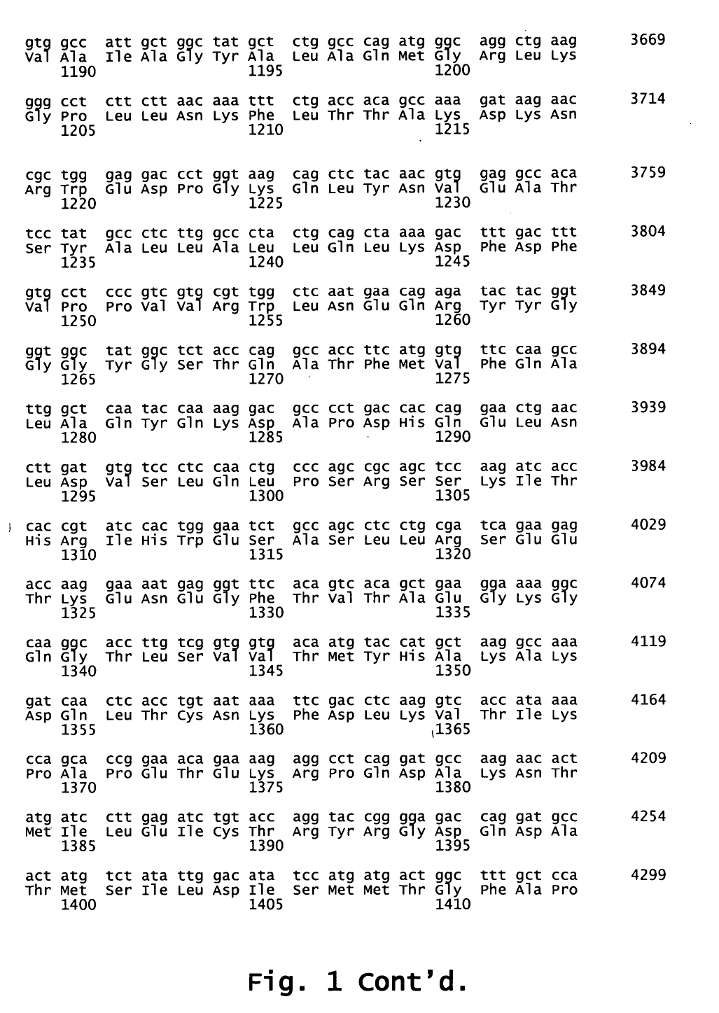

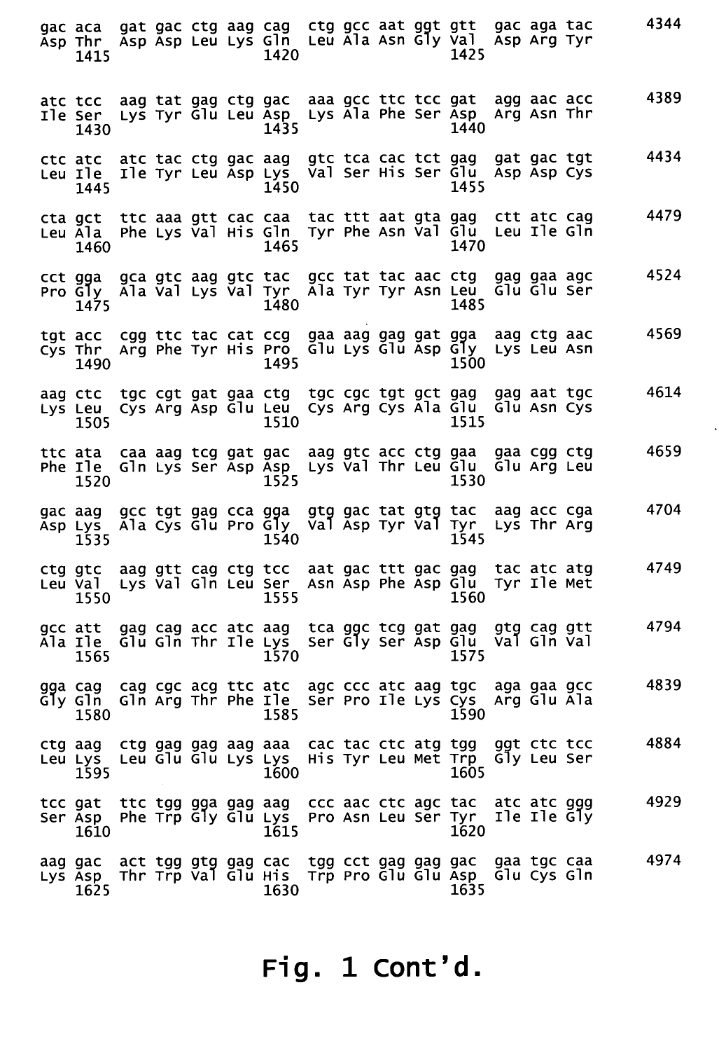

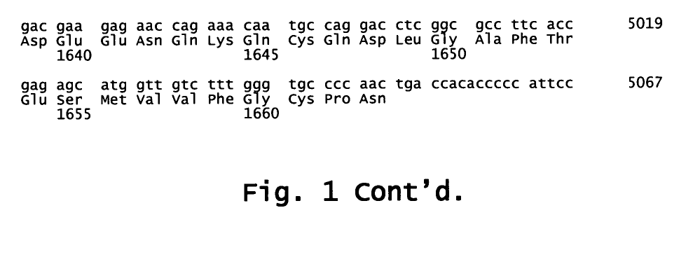

### 5. DETAILED DESCRIPTION OF THE INVENTION

The present invention is based on the finding that complement C3, its precursors and derivatives, can enhance the development of preimplantation embryos in vitro for use in assisted reproductive technologies. Specifically, supplementation of culture media with complement C3, its precursors and/or derivatives can increase the hatching rate of preimplantation embryos. Thus, it is an object of the present invention to provide methods for enhancing the development of preimplantation embryos and cells from such embryos. It is yet another object of the invention to provide an improved culture media for cultivating preimplantation embryos and cells from such embryos.

Without being bound by any theory, supplementation of culture media with complement C3, its precursors and derivatives, increases the size of the embryos and increases cell number of cell cultures as measured by cell count, diameter of blastocyst, etc. Complement C3, its precursors and/or derivatives also increase the percentage of successfully hatched blastocysts. The use of these embryos in in vitro fertilization techniques should improve the pregnancy rate and allow the use of fewer embryos, which leads to reducing the number of multiple births. These methods are generally applicable to human and veterinary uses. The methods are applicable to many species including primates and non-primates. In particular, these methods are applicable to human, bovine, canine, equine, porcine, ovine, rodent and others.

The co-culturing of oviduct cells with embryos to enhance the development of embryos has long been recognized. However, the identification of particular embryotrophic factors has been elusive. To facilitate the collection of sufficient embryotrophic factor for characterization, an immortalized human oviductal cell line, OE-E6/E7, was established that secretes the embryotrophic factor of interest (Lee et al., 2001, Mol. Reprod. Develop., 59:400-409). A human oviductal cells/mouse embryos coculture system has also been developed to screen the oviductal derived factors for their embryotrophic activity (Liu et al., 1995, Hum. Reprod., 10:2781-2786).

Three embryotrophic fractions, termed ETF-1, ETF-2 and ETF-3, were previously partially purified from human oviductal cell conditioned medium by various liquid chromatographies (Liu et al., 1995, Hum. Reprod., 10:2781-2786; Liu et al., 1998, Hum. Reprod. 13:1613-1619). They stimulate the blastulation of mouse, and enhance the implantation rate of the treated embryos (Liu et al., 1998, Hum. Reprod., 13:1613-1619). Their large molecular sizes (>100 kDa) made them unlikely to be common growth factors. They have different biological activities on mouse embryos. ETF-1 and ETF-2 stimulate the development of inner cell mass. ETF-3 enhances the development of trophectoderm cells (Xu et al., 2001, Biol. Reprod., 65:1481-1488), which leads to an increase in blastocyst size, hatching and attachment of the hatched blastocyst. While the treatment of ETF-1 and ETF-2 on embryos is effective between 24-72 hour post-hCG, ETF-3 was shown to be more effective after 72 hour post-hCG.

As shown in the Examples in Section 6, ETF-3 has been identified as a precursor of complement C3.

5.1. Complement Proteins

Complement represents a group of some 21 plasma proteins that have long been regarded as a component of the innate immune response. The complement system plays an important role, both in the resistance to infections and in the pathogenesis of tissue injury. The complement system or its components has also been implicated in maintaining feto-maternal tolerance in early pregnancy and may be involved in fertilization.

Complement C3 can be cleaved into C3a and C3b (or C3i) by one of the two complement pathways. In the classic complement pathway, C1 binds to immune complexes containing IgG or IgM antibodies. Activated C1 cleaves C2 and C4 into their active components, C2a and C4b, respectively. C2a and C4b form C3 convertase (C4b2a) which acts to cleave C3 into C3a and C3b. C3b forms a complex with C4b2a to produce C4b2a3b, which continues the complement pathway.

In the alternative pathway, C3 is cleaved to form C3b (or C3i) and combine with factor B, resulting in C3bB (or C3iB). Factor D can combine with C3bB (or C3iB) to form C3bBb, a C3 convertase capable of cleaving more C3 to C3b. This leads to more C3bBb and even more C3 conversion. C3bBb can associate with an additional C3b subunit to form the C5 convertase, C3bBbC3b. In the presence of appropriate factors (e.g., Factor I) and cofactors, C3b is further cleaved into an inactivated form, iC3b.

Thus, natural C3 derivatives can include fragments C3i, C3b, iC3b, C3a, C3c, C3d, C3dg, C3g, C3e and C3f. These fragments can be complexed to additional complement proteins which may be useful in the present invention.

Human complement C3 is an approximately 180 kDa protein encoded by a DNA sequence available at Genbank Accession NM_{—}000064. C3 is available for purchase at, for example, Research Diagnostics Inc., Flanders N.J. 07836. C3, C3b and iC3b can be purified from human serum as described in the Examples. C3, C3b and iC3b can be synthesized from recombinant means. ETF-3 can be purified and isolated as described in the Examples.

5.2. Supplementation of Culture Media

The present invention relates to culture media supplemented with complement proteins, C3, its precursors and/or derivatives. Supplementation of this component to culture media improves the in-vitro development of preimplantation embryos. In one embodiment, the present invention provides methods of increasing blastocyst size and percentage of hatched blastocysts in vitro comprising culturing a mammalian embryo in a media comprising a purified complement protein or a fragment, analog, variant or derivative thereof. In general, a complement protein, or a fragment, analog, variant or derivative thereof, is provided at a concentration of about 0.01 μg/ml to about 1000 μg/ml, in other specific embodiments, the complement protein is present at a concentration of about 0.1 μg/ml to about 100 μg/ml, preferably about 0.1 μg/ml to about 10 μg/ml, more preferably, about 1 μg/ml to about 10 μg/ml. However, any concentration that provides a physiologically significant effect can be used. A physiologically significant effect can be assessed by any means of assessing the quality of an embryo, including but not limited to, those described in Section 5.5.

Complement protein, a precursor thereof or a derivative thereof, can be used to supplement any known culture media used for the development of a preimplantation embryo, such as provided by U.S. Pat. No. 6,110,741, herein expressly incorporated by reference in its entirety.

In general, the culture medium for in vitro incubation of embryos is a composition containing inorganic salts, with or without amino acids, viatmins, purine and pyrimidine sources, energy generating cofactors, a metabolizable carbon source, a protein carrier solution. The components of the medium are selected by their known nutritive properties and on empirical studies of cell culture. The object is to provide a medium which is supportive of cell metabolism, growth and development. The objective is to mimic as closely as possible the nutritive environment of the oviduct in which naturally occurring fluids contain a variety of nutritional, hormonal, and enzymatic components.

In general, the medium for culturing embryos is a balanced salt solution, such as Synthetic Oviduct Fluid, HBSS, Ham's F10, HTF, Menezo's B2, Menezo's B3, Ham's F12, G1.2/G2.2, Earle's Buffered Salts, CZB, KSOM, BWW Medium, and emCare Media (PETS, Canton, Tex.). CZB (81.62 mM NaCl, 4.83 mM KCl, 1.18 mM KH₂PO₄, 1.18 mM MgSO₄.7H2O, 25.12 mM NaHCO₃, 1.70 mM CaCl₂.2H₂O, 31.30 mM sodium lactate, 0.27 mM sodium pyruvate, 0.11 mM EDTA, 1 mM glutamine, 5 mg/ml BSA, 100 IU/ml sodium penicillin, and 0.7 mM streptomycin) is preferred for mouse embryo culture medium, while G1.2/G2.2, HTF, B2 and B3 are preferred for human embryo culture.

In some animal systems, media have been developed which are actually much simpler than complex defined media or media containing undefined protein mixtures such as fetal calf serum. For example, U.S. Pat. No. 5,096,822, herein expressly incorporated by reference in its entirety, discloses a medium in which most standard nutrients have been omitted, including even glucose which is found to be somewhat inhibitory to bovine embryos. In fact, it appears that several ingredients in complex media may be inhibitory such as certain amino acids. Accordingly, the term “physiologically compatible” as in physiologically compatible range of concentration means levels of the substances referred to which support some cellular biochemical process known in the art, and which do not disrupt or inhibit embryonic development. The metabolizable carbon source is generally pyruvate, but lactate and/or glucose may also be used. Thus, any base or supplemented medium which is found to support embryonic development in vitro will have efficacy in the present invention so long as it incorporates a complement protein, a precursor or a derivative thereof, as an ingredient.

Additional factors may be added to the media which may improve the development of preimplantation embryos in conjunction with a complement protein. One group of supplements is growth factors, including, but not limited to, bFGF, EGF, TGF-α, TGF-β1, IGF-I, or IGF-II.

The culture media may additionally be supplemented with proteins that are expressed in the fallopian tube, including, but not limited to, Insulin-like Growth Factor 1 (IGF1), IGF2, Insulin R, IGF1 R, IGF2 R, Insulin-like Growth Factor Binding Protein 1 (IGFBP-1), IGFBP-2, IGFBP-3, IGFBP-4, IGFBP-5, IGFBP-6, Transforming Growth Factor β (TGFβ), TGFβ-RI, TGFβ-RII, Smad2/3, Inhibin α, Activin, Activin IR, Activin IIR, Follistatin, TGF-α, Epidermal Growth Factor (EGF), EGF-R, Granulocyte Macrophage-Colony Stimulating Factor (GM-CSF), GM-CSF-R, Vascular Endothelial Growth Factor (VEGF), Platelet-Derived Growth Factor (PDGF-α and -β), PDGFR-α and -β, Interleukins (IL-1α, IL-1β, IL1R t1, IL-1ra, Colony Stimulating Factor (CSF), CSF R, Leukemia Inhibitory Factor (LIF), LIF-R, IL-6, IL-6R, c-fins, Tumor Necrosis Factor α (TNF-α), TNF-R p80, TNF-R p60, c-kit, Stem Cell Factor (SCF), and gp130.

Additionally, it may be desirable to mimic the oviduct environment, by supplementing with additional components. The oviduct environment has been found to contain glucose, pyruvate, lactate, aspartic acid, glutamic acid, asparagine, serine, glutamine, arginine, glycine, threonine, alanine, tyrosine, tryptophan, methionine, valine, phenylalanine, isoleucine, leucine, lysine, sodium, chloride, potassium, calcium, magnesium. The culture media can also be optimized for pH, bicarbonate concentration and pO2.

The culture media can be provided in liquid form or as a dried powder. When provided as a dried powder, upon reconstitution with water, the concentrations of materials is equivalent to that of the liquid form.

The effective amount of a complement protein, or fragment, analog, variant or derivative thereof, contained in the culture medium will take into account, for example, whether complement 3 is used alone or whether in combination with other embryotrophic factors, the species from which the embryo is derived, the number of embryos being treated, the toxicity or undesirable effects of using an excess of complement proteins, or derivatives thereof, and other factors known to practitioners.

As a general proposition, the total pharmaceutically effective amount of each of complement protein, or a derivative thereof, in the culture medium will be at least about 0.01 μg/ml to about 1000 μg/ml of culture medium, in other specific embodiments, the complement protein is present at a concentration of about 0.1 μg/ml to about 100 μg/ml of culture medium, preferably at least about 0.1 μg/ml to about 10 μg/ml, more preferably still in the range of about 1 μg/ml to about 10 μg/ml of culture medium, although, as noted above, this will be subject to a great deal of therapeutic discretion. The key factor in selecting an appropriate dose is the result obtained, e.g., enhancement in the development of a preimplantation embryo, which can be assessed by the criteria provide above, or by other criteria as deemed appropriate by the practitioner.

5.3. Isolation of Embryos

Embryos may be obtained by in vitro fertilization (IVF) of oocytes and subsequent culture, flushing of the oviduct after fertilization and retrieval of embryos, flushing of the uterus after fertilization and retrieval of embryos, thawing of previously frozen embryos, or nuclear transfer and cloning of embryos. Cloned embryos are produced by fusing unfertilized enucleated oocytes with disaggregated cells of an existing embryo or dispersed somatic cells in order to produce multiple embryos, which are genetically identical.

Cloned embryos can also be obtained through the use of embryonic stem cells. Embryonic stem cells are ongoing cell lines of totipotent cells which came from an individual embryo. These cells are grown in a petri dish containing thousands of single cells, which, if fused with an enucleated oocyte, can lead to the production of genetically similar animals.

Alternatively, embryos can be retrieved from animals to enhance their development. In mice, isolation of embryos is described in Xu et al, 2000, Fertil. Steril., 74:1215-1219.

The methods and compositions of the present invention can also be used for culturing of embryos used to make transgenic or chimeric animals. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. See reviews of standard laboratory procedures for microinjection of heterologous DNAs into mammalian fertilized ova, including Nagy et al., MANIPULATING THE MOUSE EMBRYO, (Cold Spring Harbor Press, 2003); Costantini and Jaenisch, GENETIC MANIPULATION OF THE EARLY MAMMALIAN EMBRYO, (Cold Spring Harbor Laboratory Press, 1985); Denning and Priddle, Reproduction, 126:1-11 (2003); Wagner et al., U.S. Pat. No. 5,175,385; Krimpenfort et al., U.S. Pat. No. 5,175,384, the respective contents of which are incorporated by reference. Embryos for use in making chimeric animals can be made as described by Nagy et al., MANIPULATING THE MOUSE EMBRYO, (Cold Spring Harbor Press, 2003).

5.4. In Vitro Fertilization

In certain embodiments of the present invention, methods of in vitro fertilization are provided. In general, the steps in in vitro fertilization involve induction of ovulation, retrieval of oocytes, fertilization with sperm, cultivating the embryo and transfer of the embryo to a receptive uterus.

In inducing ovulation, hormonal regimens are adopted which increase the chances of superovulation, so that more than one oocyte reaching metaphase II is obtained. Two basic regimens involve administration of either human menopausal gonadotrophin (hMG) alone, or recombinant follicle stimulating hormone (FSH). Serum estradiol is monitored to ensure progressive increase in serum levels with simultaneous monitoring by ultrasonography of the size of the follicles.

Luteinizing hormone (LH) levels are carefully monitored, and when follicular diameter reaches about 18 mm, human chorionic gonadotrophin is administered which induces a surge in LH associated with follicular maturation and ovulation. More recently, administration of gonadotrophin releasing hormone (GnRH) agonists and antagonists have been used to suppress pituitary activity during the two week period prior to administration of chorionic gonadotrophin (CG). This has improved pregnancy rates because there is dramatic reduction in premature LH surges. For a detailed description of the use of ovarian stimulation in assisted reproduction, see Cohen J, 2002, Reproductive BioMedicine Online 6:361-366.

Recovery of oocytes in human is performed approximately 34-36 hours after administration of human chorionic gonadotrophin (hCG). This is suitably accomplished by conventional techniques, for example, using the natural cycle as described below, during surgical intervention such as oophorohysterectomy, during hormone stimulation protocols in the context of an IVF program. In the natural cycle, when the schedule of ovarian events progresses as expected, a burgeoning follicle(s) on the ovarian surface can be viewed near midcycle by ultrasound or laparoscopy, having distended vessels and substantial translucence. This is the familiar appearance of the dominant follicle near ovulation. A needle is passed into the follicle and its contents, which may be a single oocyte, are aspirated. Oocyte removal and recovery is suitably performed by means of transvaginal ultrasonically guided follicular aspiration. Following evacuation, the follicle collapses. After the follicle is aspirated, the ovum is recovered and examined microscopically to assess its condition. Additional smaller follicles may be aspirated in turn. Subjective criteria to estimate the normality of the ovum include assessing its maturity by the number and density of surrounding granulosa cells, and the presence or absence of the first polar body. Maturity of each aspirated egg is estimated by assessing the compactness of the cumulus surrounding the oocyte. Those with a loosely expanded cumulus are deemed mature, and ready for fertilization after an initial 6 hour incubation in culture media. Those oocytes deemed immature are incubated an additional 24 to 30 hours.

Spermatozoa, washed in tissue culture medium to remove seminal fluid, are further incubated in a 5% carbon dioxide atmosphere for approximately 2 hours. During this period, the sperm cells become “capacitated” as demonstrated by hyperkinetic motility. Some 10-50,000 spermatozoa are then placed in the incubation chamber with the oocytes. Fertilized eggs appear with two pronuclei 15-17 hours post-insemination. Uterine or tubal deposition via cannula is usually carried out after further 37 to 72 hour incubation until the embryo attains the four to eight cell stage. For further details of the in vitro fertilization process, see Trounson and Gardner eds., Handbook of In Vitro Fertilization, Second Edition, Washington, CRC Press, 1999

Once the oocytes are matured or stimulated to the point of being capable of fertilization, as indicated by any one or more of the factors noted above or others, they are mixed with suitable spermatozoa from the same species, resulting in fertilization. The fertilization with sperm can be carried out in vitro by known techniques including sperm injection, including those indicated below and newer technologies for effecting fertilization.

Examples of human in vitro fertilization and embryo transfer procedures that maybe successfully carried out using the method of this invention include, e.g., in vitro fertilization and embryo transfer (IVF-ET) (Quigley et al., 1982, Fertil. Steril., 38:678), blastocyst transfer (Gardner et al., 1998, Hum. Reprod., 13:3434-3440). Other suitable artificial means include, but are not limited to, in vitro fertilization and/or other artificial reproductive technologies, such as intracytoplasmic sperm injection (ICSI), subzonal insemination (SUZI), or partial zona dissection (PZD).

In IVF-ET, the oocytes are inseminated with washed and migrated spermatozoa (typically 100,000 to 200,000 per oocyte). Fertilization is assessed typically 12 to 18 hours after insemination and the oocytes are transferred to growth media such as HTF, Ham's F-10, or Earles. Only normal embryos are transferred to the patients at the 2- to 8-cell stage at typically 48 to 56 hours after retrieval or at the blastocyst stage at typically 5 days after retrieval.

General protocols for IVF include those disclosed by Trounson and Gardner eds., Handbook of In Vitro Fertilization, Second Edition, Washington, CRC Press, 1999, the disclosures of all of which are incorporated herein by reference.

Embryos may optionally be frozen prior to use in in vitro fertilization. For freezing, cryoprotective medium, generally PBS for embryos, is typically added slowly to the cells in a drop wise fashion. In addition, a cryoprotective compound is often included. Such cryoprotective compounds include permeating and nonpermeating compounds. Most commonly, DMSO, glycerol, propylene glycol, ethylene glycol, or the like are used. Other permeating agents include propanediol, dimethylformamide and acetamide. Nonpermeating agents include polyvinyl alcohol, polyvinyl pyrrolidine, anti-freeze fish or plant proteins, carboxymethylcellulose, serum albumin, hydroxyethyl starch, Ficoll, dextran, gelatin, albumin, egg yolk, milk products, lipid vesicles, or lecithin. Adjunct compounds that may be added include sugar alcohols, simple sugars (e.g., sucrose, raffinose, trehalose, galactose, and lactose), glycosaminoglycans (e.g., heparin, chrondroitin sulfate), butylated hydroxy toluene, detergents, free-radical scavengers, and anti-oxidants (e.g., vitamin E, taurine), amino acids (e.g., glycine, glutamic acid), and flavanoids and taxol (preferably 0.5-5 μm). Ethylene glycol or DMSO is preferred for embryos at a concentration range of approximately 0. 1-5%. Proteins, such as human serum albumin, bovine serum albumin, fetal bovine serum, egg yolk, skim milk, gelatin, casein or oviductin, may also be added.

The embryos to be frozen are aspirated into cryovials or straws and placed in the vapor phase of liquid nitrogen for one to two hours, and then plunged into the liquid phase of liquid nitrogen for long-term storage or frozen in a programmable computerized freezer. Frozen embryos are thawed by warming in a 37° C. water bath and are directly transferred to the patient or cultured to blastocyst stage before embryo transfer. Other cooling and freezing protocols may be used. Vitrification involves dehydration of the embryos using sugars, Ficoll, or the like. The embryo is then added to a cryoprotectant and rapidly moved into liquid nitrogen.

5.5. Measurement of Enhanced Development of Preimplantation Embryos

Once cultured, enhanced development of preimplantation embryos can be measured using standard assays. In the methods of the present invention, it is desirable to culture the preimplantation embryos until the blastocyst stage.

Embryonic development may be evaluated by a variety of tests including normal cleavage or division of the embryo in culture; normal formation of a blastocyst cavity at an appropriate time in culture; counting the number and health of cells found in the embryo; transfer to a female and establishment of a pregnancy; and subsequent birth of a normal offspring.

In general, embryos can be classified into by their developmental stage, e.g., four-cell to seven-cell, eight-cell to 16-cell, expanded blastocyst and hatching blastocyst. After the 8-cell or 16-cell embryo stage (depending on the species), a morula, i.e., a mulberry-shaped mass of cells, is formed. Membrane transport molecules are expressed which result in an accumulation of fluid inside the embryo and signals formation of the blastocyst. A blastocyst is composed of two distinct tissues, a hollow sphere of trophoblast cells, inside of which is a small cluster of cells called the inner cell mass. The blastocyst undergoes hatching in which the inner cell mass escapes. The hatching blastocyst has a clear herniation of the zona pellucida by the trophectoderm (TE). When in vivo, the hatched blastocyst undergoes implantation or elongates rapidly to fill the uterine lumen.

One means of assessing development of embryos is by morula or blastocyst formation. In one method, the percentage of embryos in these stages is assessed. Embryos can be classified by any means, including, but not limited to examination under a light microscope. Staining methods are described in Xu et al., 2000, Fertility and Sterility 74:1215-1219. In another method, the cell count of embryos at the blastocyst stage are used. Cell counts can be taken as described by Liu et al. (1995, Human Reproduction 10:2781-2786).

Another means for measuring enhanced development of embryos is by assessing blastocyst size or hatching blastocysts as described in Xu et al. 2001, Biol Reprod 65; 1481 -1488. Images of blastocysts can be taken by a digital camera (Photometrics, Tucson, Ariz.) for analysis by MetaMorph Imaging System (Universal Imaging, West Chester, Pa.). Blastocysts are transferred into 20 ml droplets of DMEM/F12 containing 15% human serum under paraffin oil. The number of embryos attached to the culture dish is counted 2 days later. The MetaMorph Imaging System is used to determine the spreading area of blastocyst outgrowth. The percentage of hatched blastocysts may be determined from the total number of developing embryos.

Yet another means for measuring enhanced development of embryos is by assessing the degree of blastomere fragmentation. The perivitelline space is examined and <25% fragments is classified as mildly fragmented, while >25% fragments is classified as severely fragmented.

Yet another means for measuring enhanced development of embryos is by assessing the degree of apoptosis prevention. Embryos can be stained by TUNEL (In situ cell death detection system; Boehringer Mannheim, Germany) as described in Xu et al., 2000, Fertil. Steril., 74:1215-1219.

Yet another means for measuring enhanced development of embryos is by measuring mitochondrial transmembrane potential as described in Xu et al., 2003, Fertil. Steril., 80:178-183.

Yet another means for measuring enhanced development of embryos is by measuring capsase activity as described by Xu et al., 2001, Fertil. Steril., 75:986-91.

The invention will be more fully understood by reference to the following examples. They should not, however, be construed as limiting the scope of the invention. All literature and patent citations are expressly incorporated by reference.

### 6. EXAMPLES

6.1. Purification of ETF-3 from Human Oviductal Cell Conditioned Medium

Conditioned media derived from primary human oviductal cells and immortalized oviductal cells, OE-E6/E7 was fractionated as described in Liu et al., 1998, Hum. Reprod., 13:1613-1619. OE-E6/E7 cells were grown in Dulbecco modified Eagle medium/Ham F12 (DMEM/F12) supplemented with 0.3% (w/v) BSA (Sigma, St. Louis, Mo.). Fifty ml of oviductal cell-conditioned medium was passed through a concanavalin A column using a fast-performance liquid chromatographic system (Amersham Pharmacia Biotech, Uppsala, Sweden). The column was washed with a start buffer (20 mM Tris [pH 7.4], 0.5 M NaCl, 1 mM MgCl₂, 1 mM CaCI₂, and 1 mM MnCl₂) at a flow rate of 0.3 ml/min for 30 minutes to remove unbound molecules. The bound glycoproteins were eluted with the same buffer containing 0.3 M α-D-methylglycoside at a flow rate of 0.3 ml/min, concentrated by ultrafiltration through the Centricon-100 (Amicon, Inc., Beverly, Calif.), and further fractionated by a Mono-Q column using the SMART System (Amersham Pharmacia Biotech). The ETF-3 was eluted from the column with 20 mM Tris-HCl (pH 7.5) containing 0.3 M NaCl at a flow rate 70 μl/min. The purified fraction was desalted, concentrated by the Centricon-100, and reconstituted with the appropriate medium.

6.2. Production of Monoclonal Antibody Against ETF-3

In order to purify ETF-3 to homogeneity, monoclonal antibodies against ETF-3 were raised. Anti-ETF-3 monoclonal antibodies were generated by immunizing Balb/cByJ mice subcutaneously with 100 μg affinity-purified ETF-3 proteins isolated from oviductal cell conditioned medium in 200 μl of emulsion containing equal volume of PBS and complete Freund's adjuvant (Sigma) (Day 0). Booster doses were similarly given in Freund's incomplete adjuvant on day 28 and 42. Three days prior to fusion, a final injection of ETF3 in sterile PBS was given through tail vein. Fusion of spleen cells and mouse plasmacytoma Sp2/0 cells was carried out as described previously (Luk et al., 1990, J. Immunol. Methods, 129:243-250), using polyethyleneglycol PEG4000 (Sigma Co., St. Louis, Mo.) as fusion agent. Hybridomas were cultivated and selected in RPMI 1640 (Gibco BRL, Paisley, Scotland) standard medium, containing 10% heat-inactivated fetal bovine serum and HAT (0.1 mM hypoxanthine, 0.016 mM thymidine and 0.4 μM aminopterine) supplemented with 1 mM sodium pyruvate, 100 U penicillin-streptomycin and 5 mM L-glutamine.

Screening was performed by enzyme immunoassay 10-12 days after fusion against homologous purified ETF-3 antigen. Positive clones were tested against a battery of different antigens before cloning by limiting dilution. Cloned hybridomas were grown in serum-free medium or ascites fluid.

The hybridoma clones producing conditioned medium with the highest immunoreactivity were allowed to proliferate. Two to three liters of the hybridoma condition medium was allowed to pass through a 5-ml HiTrap Protein G column (Amersham Pharmacia Biotech) and washed with a start buffer (20 mM sodium phosphate, pH 7.0). The bound antibody was eluted with elution buffer (0.1 M glycine-HCl, pH 2.7) and collected in a neutralizing solvent (1M Tris-HCl, pH 9.0) to bring the final pH to neutral. The purified antibody was concentrated by the Centricon-30 (Amicon, Inc., Beverly, Calif.). The immunoreactivity of the purified monoclonal antibody was confirmed by western blot analysis of three different batches of ETF-3.

The conditioned media derived from immortalized oviductal cells, OE-E6/E7, were fractionated as previously described (Lee et al., 2001, Mol. Reprod. Develop., 59:400-409). Briefly, the cells were grown in Dulbecco modified Eagle medium/Ham F12 (DMEM/F12) supplemented with 0.3% (w/v) BSA (Sigma, St. Louis, Mo.). Fifty milliliters of oviductal cell conditioned medium were passed through a concanavalin-A column using a fast-performance liquid chromatographic system (Amersham Pharmacia Biotech, Uppsala, Sweden). The column was washed with a start buffer (20 mM Tris, 0.5 M NaCl, 1 mM MgCl₂, 1 mM CaCl₂, and 1 mM MnCl₂, pH 7.4) at a flow rate of 0.3 ml/min for 30 min to remove unbound molecules. The bound glycoproteins were eluted with the same buffer containing 0.3 M α-D-methylglucoside at a flow rate of 0.3 ml/min. The eluate was dialyzed against PBS in porous tubing with molecular size cutoff of 12-14 kDa (Spectrum Laboratories, Inc. Rancho Dominguez, Calif.). Two hundred microgram of purified monoclonal antibody (clone 14) was added to the dialysed concanavalin-A eluate and incubated overnight at 4° C. on a rocking platform. The mixture was allowed to pass through a 5-ml HiTrap Protein G column (Amersham Pharmacia Biotech) and washed with a start buffer (20 mM sodium phosphate, pH 7.0). The bound antibody-antigen complex was eluted with elution buffer (0.1 M glycine-HCl, pH 2.7) and collected in a neutralizing solvent (1 M Tris-HCl, pH 9.0) to bring the final pH to neutral. The purified fraction was concentrated by the Centricon-30 (Amicon, Inc., Beverly, Calif.).

To confirm that the protein-G column can purify ETF-3 from concanavalin-A bound fraction of OE-E6/E7 conditioned medium, the concentrated eluent and CM in 1×SDS protein sample buffer (50 mM Tris-HCl; pH 6.8, 2% SDS, 0.1% bromophenol blue, 10% glycerol, 1% β-mercaptoethanol) were denatured for 5 min at 95° C., fractionated by 8% SDS-PAGE. Western Blot was performed by transferring the protein onto PVDF membrane. The membrane was blocked with 5% skim milk in PBST (0.05% Tween 20), and probed with purified clone 14 antibody with 1:100 dilution in blocking solution overnight at 4° C. The membrane was further washed 5 times with PBST for 5 min at room temperature, and incubated with anti-mouse IgG antiserum conjugated with horseradish peroxidase (1:5000 in PBST). After 1 hr of incubation, the membrane was washed thrice and visualized by enhanced chemiluminescence (ECL) according to the manufacturer's recommendations (Santa Cruz, Calif.).

About 10 μg of protein-G affinity purified ETF-3 was reconstituted in 250 μl of rehydration solution (8 M urea, 4% w/v CHAPS, 2% v/v pharmalyte 3-10, 0.002% bromophenol blue), and loaded onto a 13 cm IPG strip in Ettan™ IPGphor™ Strip Holder (Amersham Pharmacia Biotech, Uppsala, Sweden) for overnight in-gel rehydration. The first dimension was run on a Ettan™ IPGphor™ isoelectric Focusing System (Amersham Pharmacia Biotech) for a total of 16000 kVh at 20° C. The iPG strip was then placed in the equilibration buffer (2% SDS, 50 mM Tris-HCl, pH 8.8, 6 M urea, 30% v/v glycerol, 0.002% bromophenol blue) containing 10 mg/ml DTT for 15 min, followed by another 15 min in equilibration buffer containing 25 mg/ml of iodoacetamide. A ten percentage slab gel (10% acrylamide, 0.27% N,N′-methylenebisacrylamide, 375 mM Tris-HCl, pH 8.8, 0.1% SDS, 0.05% ammonium persulfate, 0.005% TEMED) was casted using Hoefer SE 600 system. The IPG strip was then sealed onto the SDS slab gel with 0.5% agarose in SDS electrophoresis buffer (25 mM Tris-HCl, pH 8.3, 192 mM glycine, 0.1% w/v SDS). The second dimension was performed in the electrophoresis buffer at 15 mA/gel for 15 mins and followed by 5 hours at 30 mA/gel.

After overnight fixation in 40% ethanol/10% acetic acid, sensitization of the gel was performed using 0.2% w/v sodium thiosulfate and 30% ethanol for 1 hr followed by five 8 min washes in doubled distilled water. The gel was then incubated in 0.25% silver nitrate for 1 hr, briefly washed in doubled distilled water and finally developed with a solution containing 2.5% sodium carbonate and 0.015% formaldehyde (37%) until a clear image was seen. Staining was stopped by rinsing the gel with 5% acetic acid.

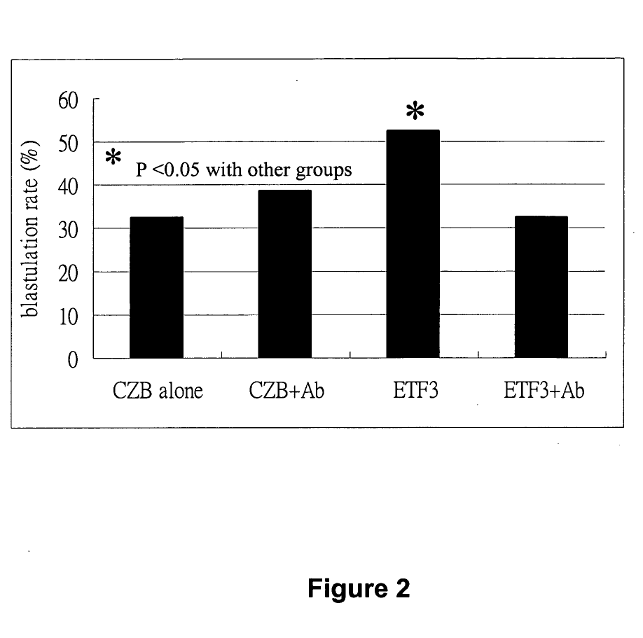

The antiserum of the mouse from which the hybridoma clones was raised nullifies the activity of ETF-3 on blastulation and hatching of mouse embryos (Lee et al., 2003, Biol. Reprod., 68:375-382). The antiserum also binds to the epithelial cells of human fallopian tube and to the blastomeres of ETF-3 treated mouse embryos (Lee et al., 2003, Biol. Reprod., 68:375-382). The monoclonal antibody from hybridoma (clone-14) abolishes the embryotrophic effect of ETF-3 (FIG. 2).

6.3. Identification of ETF-3 by Mass Spectrometry

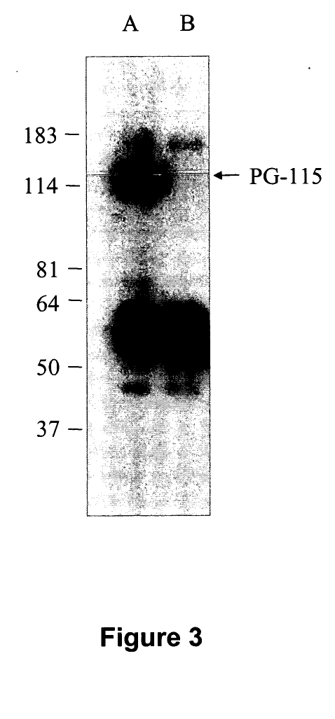

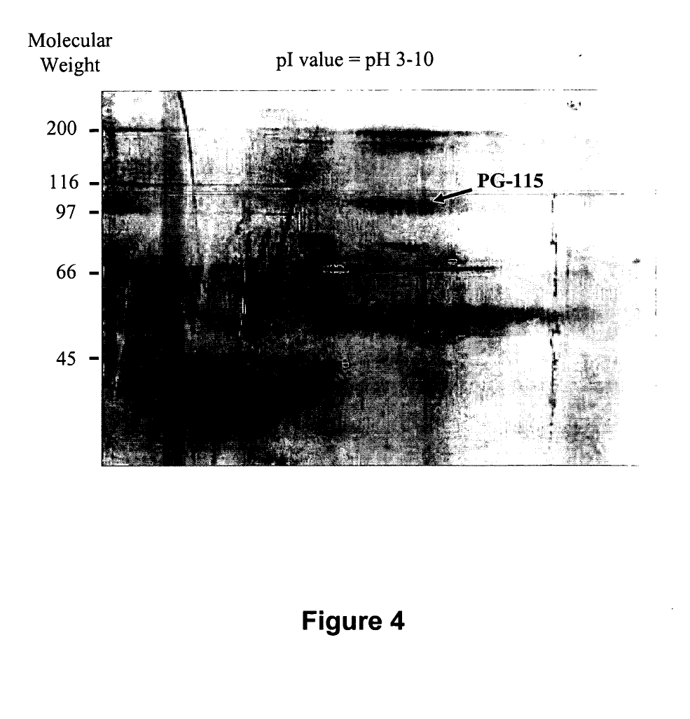

Western blot analysis of protein-G purified anti-ETF-3 antibody and ETF-3 immunocomplex showed the presence of an extra 115 kDa protein (PG-115, lane A, FIG. 3) when compared with that of antibody alone (lane B, FIG. 3). The protein (arrow in FIG. 4) had a pI value of about 6-7 when the immunocomplex was subjected to two-dimensional gel electrophoresis.

The 115-kDa protein band from one-dimensional SDS-PAGE of anti-ETF-3 antibody affinity purified ETF-3 was used for mass spectrometry analysis. The excised gel was sliced into 1×1 mm pieces and equilibrated with 0.5 ml of 50 mM NH₄HCO₃ for 10 min with 700 rpm agitation at room temperature. The gel was destained by washing twice with 500 μl of 50% acetonitrile in 50 mM NH₄HCO₃ for 30 mins, washed with acetonitrile and subjected to complete dryness using a centrifugal vacuum concentrator. In gel digestion was performed using 15 μl of trypsin solution (10 μg/ml trypsin in 25 mM NH₄HCO₃, pH 8.0) overnight at 37° C.

About 10% of the digest was analyzed on a Voyager-DE™ STR MALDI-TOF mass spectrometer (PerSeptive Biosystems, Framingham, Mass., USA). Briefly, 2 μl of the digested sample was applied onto the sample plate and allowed to dry to 1 μl before adding with 1 μl of matrix solution (4 mg/ml α-cyano-4-hydroxycinnamic acid in 35% acetonitrile and 1% trifluoroacetic acid). The spot was dried at room temperature and subjected to mass analysis. The MS mass spectrum was obtained in delayed extraction mode using an accelerating voltage of 25 kV and 175 nsec delay. Trypsin-peptide mass to charge ratios of 906.5049, 1153.5741 and 2163.0570 were used for internal calibration. Database searching was performed with ProteinProspector MS-Fit (http://prospector.ucsf.edu).

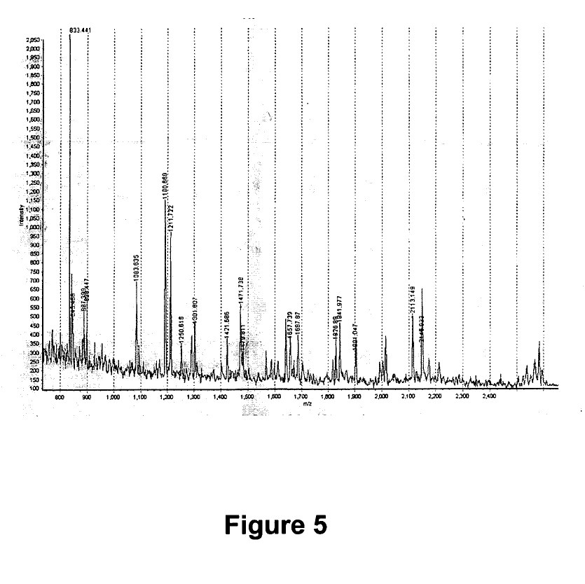

The MALDI-TOF mass spectrum of the protein after in-gel tryptic digestion is shown in FIG. 5. Database searches showed that the peptide mass fingerprint was complement C3 precursor (Table 1).

TABLE 1

Identification of protein-G purified 115 kDa protein by
MALDI-TOF mass spectrometry

| | | | | | | |
|---|---|---|---|---|---|---|
| | Mass | Mass | | | | SEQ ID |
| Fraction | submitted | matched | Start | End | Peptide sequence | NO: |

| | | | | | | |
|---|---|---|---|---|---|---|
| 1 | 833.4410 | 833.4885 | 835 | 841 | LPYSVVR | 3 |
| 2 | 845.4060 | 845.4270 | 1255 | 1260 | WLNEQR | 4 |
| 3 | 887.3990 | 887.4587 | 842 | 848 | NEQVEIR | 5 |
| 4 | 898.4470 | 898.4787 | 849 | 855 | AVLYNYR | 6 |
| 5 | 1083.6350 | 1083.5587 | 1052 | 1060 | GYTQQLAFR | 7 |
| 6 | 1190.6690 | 1190.6210 | 1245 | 1254 | DFDFVPPVVR | 8 |
| 7 | 1211.7220 | 1211.6537 | 1311 | 1320 | IHWESASLLR | 9 |
| 8 | 1421.6860 | 1421.6371 | 1451 | 1462 | VSHSEDDCLAFK | 10 |
| 9 | 1471.7380 | 1471.7446 | 914 | 926 | AAVYHHFISDGVR | 11 |
| 10 | 1657.7390 | 1657.7644 | 1172 | 1185 | AGDFLEANYMNLQR | 12 |
| 11 | 1687.8700 | 1687.8478 | 1186 | 1201 | SYTVAIAGYALAQMGR | 13 |
| 12 | 1841.9770 | 1841.9914 | 1463 | 1478 | VHQYFNVELIQPGAVK | 14 |
| 13 | 2146.9330 | 2147.0733 | 1285 | 1303 | DAPDHQELNLDVSLQLPSR | 15 |

Protein sequence of human complement component 3 precursor as retrieved from Protein Prospector MS-Fit software search result (http://prospector.ucsf.edu) (accession no. NM_000064, NCBI Entrez).

6.4. Comparison of C3, its Fragments and ETF-3 by Western Blot Analysis

To confirm ETF-3 secreted from OE-E6/E7 cells was actually C3 and the monoclonal antibody raised against ETF-3 also recognized C3 fragment, different C3 fragments (C3, C3b and iC3b) purified from human serum (Calbiochem, Calif., USA) were separated by 8% SDS-PAGE. Concanavalin-A eluted fraction and ETF-3 derived from OE-E6E7, conditioned medium of OE-E6E7 before and after Centricon-100 centrifugation were also included in the gel. After SDS-PAGE, the proteins were transferred to PVDF membrane, on which Western blot were performed using goat anti-human C3 antibody (1:20,000 dilution in blocking solution containing 5% skim milk in PBST) overnight at 4° C. The membrane was then successively washed 5 times with PBST for 5 min at room temperature, incubated with anti-goat IgG conjugated with horseradish peroxidase (1:5000) for 1 hr, washed 5 times with PBST and visualized by ECL technique (Amersham Uppsala, Sweden). The protein bands in all the samples were observed with Coomassie Blue staining.

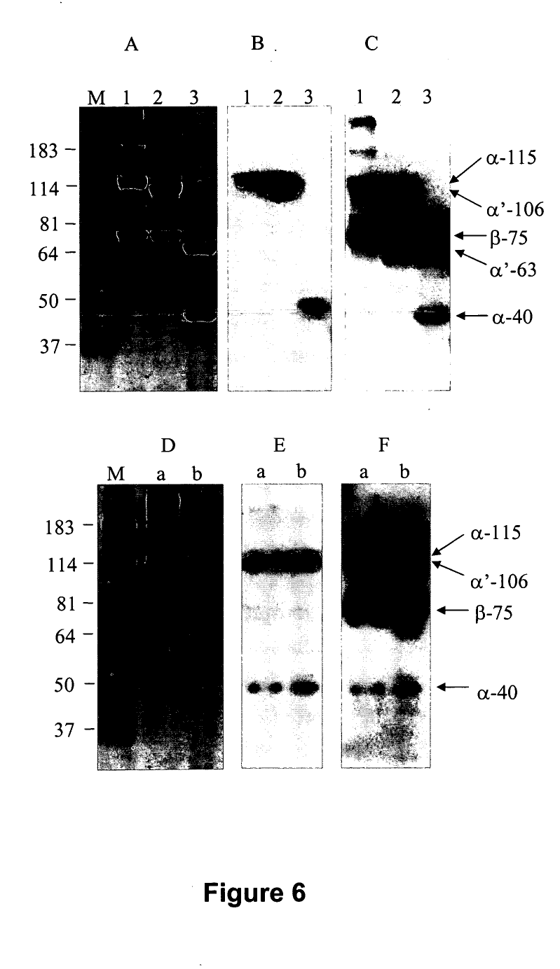

FIG. 6A shows the protein patterns of commercially available C3 and its fragments, C3b and iC3b. Polyclonal antibody against C3 recognized all the subunits in C3 and its fragments (FIG. 6C). Anti-ETF-3 antibody (clone 14) reacted with the 115 kDa α-chain of C3 (α-115) and the 106 kDa α′-chain of C3b (α′-106), and the 40 kDa α-chain of iC3b (α-40) (FIG. 6B), indicating that the binding epitope of the antibody was in the α-40 of C3 fragments.

Anti-C3 polyclonal antibody detected the presence of C3 fragments in purified and partially purified ETF-3 (concanavalin-A bound fraction of OE-E6/E7 conditioned medium) with sizes of 115, 106, 75 and 40 kDa (FIG. 6F). Mass spectrometry analysis of the 115, 106 and 75 kDa bands confirmed their identities as C3 precursor. A band of 190 kDa was also found in purified ETF-3 and partially purified samples. This possibly represented C3 precursor in the lysate of OE-E6/E7 that had been concentrated after purification. Anti-ETF-3 antibody recognized the 115, 106, and 40 kDa bands of purifed and partially purified ETF-3 (FIG. 6E).

6.5. Immunohistochemical Staining of Human and Mouse Oviduct and OE-E6/E7

Human fallopian tube tissue was obtained from patients admitted for tubal ligation or hysterectomy due to uterine fibromyoma. Six micrometers thick formalin-fixed and paraffin-embedded human oviduct tissue sections were placed on slides, warmed at 42° C. overnight and 65° C. for 30 mins, and subsequently dewaxed in xylene. After rehydration through a graded series of ethanol, the sections were washed with PBS, rehydrated and permeabilized using microwave at high power for 3 min and low power for 17 min in Target Retrieval Solution (Dako. Calif.). After cooling, the sections were washed 3 times with PBS for 5 min each time. The sections were incubated in the blocking solution (10% normal rabbit serum in PBS) for 2-4 hours at RT and subsequently with the primary antibody, anti-C3 (Calbiochem, Germany) diluted at 1:2000 in blocking solution at 4° C. overnight in a humidified chamber. Following eight washes with PBS, 10 min for each wash, all the sections were incubated with the second antibody, fluorescein isothiocyanate-labeled rabbit anti-goat IgG antibody (1:100 [v/v] in 10% normal rabbit serum in PBST) in a dark humidified incubator at 37° C. for 1 hr, rinsed six times with PBST, counterstained with propidium iodide (PI), and mounted with Fluorescent mounting medium (Dako, Calif.) after washing of the excess PI. The negative control sections were incubated in parallel with omission of the first antibody or with OE-E6/E7 derived ETF-3 or iC3b preabsorbed anti-C3 antibody. The sections were observed with a confocal microscope.

The presence of C3 in MF 1 mouse oviducts at met-estrus stage was also studied by immunohistochemistry using polyclonal antibody against the β-chain of C3 (Santa Cruz, Calif.). Negative control was obtained by omitting the primary antibody on mouse oviduct. The sections were observed under a fluorescent microscope.

OE-E6/E7 were seeded in a chamber slide (Nunc, Inc., Naperville, Ill.) and cultured with serum supplemented DMEM/F12. Two days later, the cell layers were rinsed with 0.1% Tween-20/PBS (PBST), fixed with 4% (v/v) paraformaldehyde in PBS (pH 7.35) for 30 min, permeated with 0.1% (v/v) Triton X-100/PBS on ice for 1 min, and rinsed six times with PBST. The cells were then blocked in 10% (v/v) normal rabbit serum/PBST at room temperature for 2 hours, incubated with anti-C3 antibody (1:2000 [v/v] in 10% normal rabbit serum/PBST) at 4° C. overnight, and washed 6 times with PBST. C3 and OE-E6/E7 derived ETF-3 (antigen to antibody ratio was 5:1 [w/w]) were used to pre-absorb anti-C3 at 4° C. overnight as negative control. Fluorescein isothiocyanate-labeled rabbit anti-goat IgG antibody (1:100 [v/v] in 10% normal rabbit serum in PBST) was then incubated with the cells in a dark humidified incubator at 37° C. for 1 hr and rinsed six times with PBST. The cell nuclei were counterstained with 10 μg/ml PI in PBS. The cells were then observed with a fluorescent microscope.

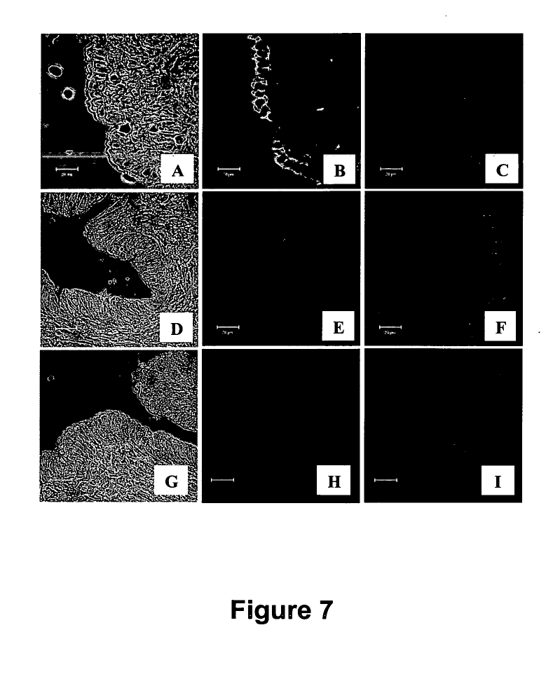

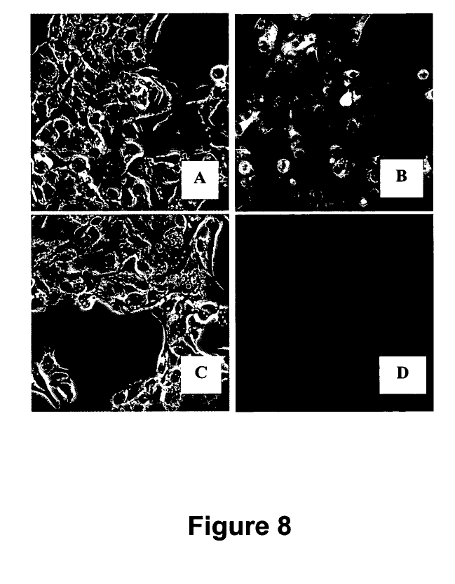

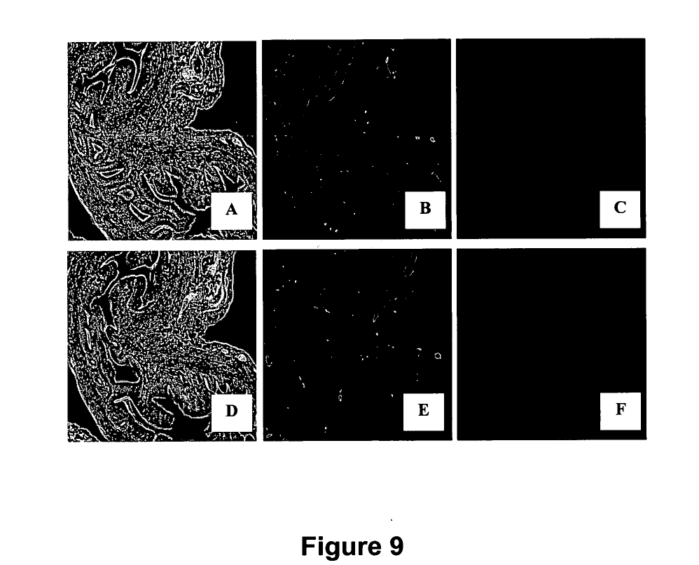

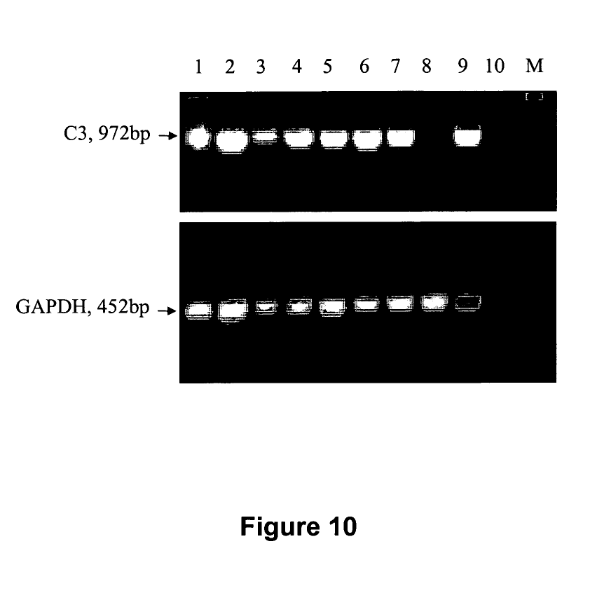

C3 immunoreactivity was localized to the epithelial lining of human fallopian tubes obtained from patients admitted for tubal ligation or hysterectomy due to uterine fibromyoma (FIG. 7). After pre-absorption of the anti-C3 antibody with ETF-3 or iC3b, no signal was found in the sections. Immortalized oviductal cells, OE-E6/E7 possessed C3 immunoreactive signal, which was absent when anti-C3 antibody pre-absorbed with ETF-3 was used (FIG. 8). Positive staining of C3 in the epithelial lining of mouse oviduct at metestrus stage was also found (FIG. 9).

6.6. mRNA Expression of C3 in Oviductal Cells

Total RNAs from OE-E6/E7 cells at passages 14 and 25, the primary oviductal cell OE 89 and OE 109, human oviductal epithelium tissues, human liver tissue, SKOV-3 and CHO-K1 cell lines were isolated using Trizol Reagent (Invitrogen, Carlsbad, Calif.) according to the manufacturer's protocol. The quantity and quality of total RNA samples were analyzed by UV spectrophotometry. One hundred nanogram of total RNA was subjected to RT-PCR using the Access RT-PCR System (Promega, Madison, Wis.). In brief, the samples were incubated at 48° C. for 45 min for first strand cDNA synthesis. PCR amplification was carried out for 40 cycles at 94° C. for 30 sec, 60° C. for 1 min and 68° C. for 2 min usig gene-specific primers of human C3 (5′-GGTCAAGCAGGACTCCTTGT-3′; SEQ ID NO:16 and 5′-CCCTTGTTCATGATGAGGTAG-3′; SEQ ID NO:17) to generate a 972-bp DNA fragment and GAPDH (5′-ACCACAGTCCATGCCATCAC-3′; SEQ ID NO:18 and 5′-TCCACCACCCTGTTGCTGTA-3′; SEQ ID NO:19) to generate a 452-bp DNA fragment. The PCR products were analyzed in a 2% agarose gel (Gibco BRL).

Human C3 transcripts were obtained in cultured primary OE cell from patient number 109 (lane 1), 89 (lane 2), immortalized OE-E6/E7 at passages 14 (lane 3), 25 (lane 4), human oviductal epithelium tissue from 2 different patients (lane 5 and 6), SKOV-3 cell (lane 7) and human liver tissue (lane 9). No C3 was found in CHO-K1 cell (lane 8) and dH₂O control (lane 10).

6.7. Embryotrophic Activity of C3 and its Fragments

The protocol for obtaining mouse embryos was approved by the Committee on the Use of Live Animal for Teaching and Research, the University of Hong Kong. Mature MF1 female mice (age, 6-8 wk) were superovulated with 5 IU of equine chorionic gonadotropin (eCG; Sigma), followed by an injection of 5 IU of hCG (Sigma) 46 h later. The MF1 female mice were mated with proven-fertile BALB/c males and the day with the presence of vaginal plug was regarded as Day 1. The zygotes were recovered 24 h post-hCG from the oviductal ampullae into Hepes-buffered CZB (CZB/HEPES) (Chatot et al., 1989, J. Reprod. Fertil., 86:679-688) containing 0.8 mg/ml of hyaluronidase (Sigma) to remove the cumulus mass. They were washed three times in 250 μl of CZB/HEPES, followed by one wash in CZB, before being pooled and allocated randomly in groups of 20-30 for culturing in CZB alone, CZB supplemented with 10 μg/ml of C3, C3b and iC3b (Calbiochem) for the first 48 h. They were then transferred to CZB containing 5 mM glucose (CZB+G) and appropriate supplementation of ETF-3, C3 or its fragments.

The percentages of embryos reaching fully expanded blastocyst and hatching blastocyst were recorded at approximately 120 and 144 hr post-hCG, respectively. The image of each expanded blastocyst was captured with a phase-contrast inverted microscope. The area of each expanded blastocyst was determined using the MetaMorph imaging system (version 3.51; Universal Imaging Corp., West Chester, Pa.) and compared among different C3- fragment treatment and medium-alone culture groups. The data obtained from four batches of mouse embryo were combined and analyzed by Chi-square test or Student t-test where appropriate.

The development of mouse embryo in medium alone and medium supplemented with different C3 fragments is shown in Table 2. The rate of embryo development was based on the number of 2-cell embryos after 24 hours of culture. The embryos incubated with iC3b for 4 days had significantly more expanded blastocysts and higher hatching rate (p<0.05) when compared with those cultured in medium alone and media supplemented with other C3 fragments. The size of the expanded blastocysts in the iC3b group as determined by the area of the expanded blastocyst was also significantly larger than other groups (p<0.05). Both C3b and iC3b stimulated hatching of the treated embryos.

TABLE 2
Effects of C3 and its fragments on mouse embryo development in vitro

| | No. of | Expanded Blastocyst | | Hatching |
|---|---|---|---|---|
| | 2-cell Embryo | No. (%) | Area (μm²) | Blastocyst, n (%) |
| CZB | 230 | 128 (56)^{a} | 9265 ± 170^{a} | 51 (22)^{e} |
| C3 | 129 | 69 (53)^{a} | 8961 ± 208^{a} | 35 (27)^{g} |
| C3b | 140 | 85 (61) | 9514 ± 223^{a} | 49 (35)^{f} |
| iC3b | 131 | 88 (67)^{b} | 11171 ± 245^{b} | 58 (44)^{h} |

^{a-b,e-f,e-h,g-h}p < 0.05 for values within the same column

The embryotrophic activity of iC3b was also found in another mouse strain, ICR x BALB/c. When the embryos from this strain were cultured in different culture media, i.e., CZB, potassium-simplex optimized medium supplemented with amino acids (KSOMaa; Specialty Media, Phillipsburg, N.J.) or Gardner's medium version 3 (G1/G2 v3; Vitrolife, Sweden), the supplementation of iC3b enhanced blastulation and increased the size of the resulting blastocysts (Table 3).

TABLE 3
Effects of iC3b on ICR × BALB/c mouse embryo development

| | | Expanded Blastocyst | |
|---|---|---|---|
| Culture Media | No. of 2-cell Embryo | No. (%) | Area (μm²) |
| CZB | 204 | 71 (35) | 8252 ± 120 |
| CZB + iC3b | 210 | 99 (47)* | 8943 ± 114* |
| G1/G2 v3 | 142 | 46 (32) | 9440 ± 217 |
| G1/G2 v3 + iC3b | 186 | 99 (54)* | 10352 ± 217 |
| KSOMaa | 144 | 43 (30) | 9558 ± 182 |
| KSOMaa + iC3b | 162 | 68 (42)* | 10716 ± 159* |

*iC3b-treated groups were significantly different from the corresponding control groups cultured in medium alone (P < 0.05).

6.8. Conclusion

Four observations showed that the ETF-3 is a complement C3 derivative. The observations were: (1) monoclonal anti-ETF-3 antibody that neutralizes the embryotrophic effect of ETF-3 recognized a 115 kDa protein identified as C3 precursor by mass spectrometry; (2) anti-ETF-3 antibody and commercially available C3 antiserum react with C3, C3 fragments and ETF-3 in Western blot analysis; (3) the immunoreactive signal of C3 was localized to the epithelial cells of human oviduct and immortalized oviductal cells and could not be detected after preabsorption of the anti-C3 antiserum with ETF-3 and iC3b; and (4) supplementation of iC3b and C3b to the culture medium stimulated the development of mouse blastocyst in terms of size and hatching. This last observation is consistent with previous observations of increased hatching rate of human embryos after oviductal cell coculture (Yeung et al., 1992) and improved trophectoderm development of mouse embryos after ETF-3 treatment (Xu et al., 2001) and indicate that the use of the complement or its fragments can improve embryo development in vitro.

All references cited herein are specifically incorporated by reference as if fully set forth herein.

Having hereinabove disclosed exemplary embodiments of the present invention, those skilled in the art will recognize that this disclosure is only exemplary such that various alternatives, adaptations, and modifications are within the scope of the invention, and are contemplated by the Applicant. Accordingly, the present invention is not limited to the specific embodiments as illustrated above, but is defined by the following claims.

1. A method for developing a preimplantation mammalian embryo in vitro comprising:
a) culturing a mammalian embryo in a medium comprising a purified complement C3 protein, a fragment thereof, precursor thereof, an analog thereof, or derivative thereof; and
b) developing the embryo to the blastocyst stage.

2. The method of claim 1, wherein the C3 protein is selected from the group consisting of ETF-3, C3, C3i, C3a, C3b, iC3b, C3c, C3d, C3dg, C3g, C3e and C3f.

3. The method of claim 1, wherein the C3 protein is present at a concentration of about1 0.01 μg/ml to about 1000 μg/ml.

4. The method of claim 3, wherein the C3 protein is present at a concentration of about 0.1 μg/ml to about 100 μg/ml.

5. The method of claim 4, wherein the C3 protein is present at a concentration of about 1 μg/ml to about 10 μg/ml.

6. The method of claim 1, wherein the C3 protein is present at a physiological concentration that enhances the development of mammalian embryos relative to the development of embryos cultured in a medium without the C3 protein.

7. The method of claim 1, wherein the mammalian embryo is a primate embryo.

8. The method of claim 7, wherein the primate embryo is a human embryo.

9. The method of claim 1, wherein the mammalian embryo is a non-primate embryo derived from a non-primate selected from the group consisting of canines, felines, mouse, bovines, sheep and pigs.

10. A method for in vitro fertilization comprising:
a) obtaining oocytes from a female donor;
b) incubating the oocytes in a culture medium;
c) fertilizing in vitro the oocytes with sperm to produce at least one fertilized oocyte;
d) culturing the fertilized oocyte to produce an embryo in a medium comprising
a complement C3 protein, a precursor thereof, a fragment thereof, an analog thereof, or a derivative thereof; and
e) transferring at least one embryo to the uterus of a mammal.

11. The method of claim 10, wherein the C3 protein is selected from the group consisting of ETF-3, C3, C3i, C3a, C3b, iC3b, C3c, C3d, C3dg, C3g, C3e and C3f.

12. The method of claim 10, wherein the C3 protein is present at a concentration of about 0.01 μg/ml to about 1,000 μg/ml.

13. The method of claim 12, wherein the C3 protein is present at a concentration of about 0.1 μg/ml to about 100 μg/ml.

14. The method of claim 13, wherein the C3 protein is present at a concentration of about 1 μg/ml to about 10 μg/ml.

15. The method of claim 10, wherein the C3 protein is present at a physiological concentration that enhances the development of mammalian embryos relative to the development of embryos cultured in a medium without the C3 protein.

16. A composition comprising a culture medium comprising a complement C3 protein, a precursor thereof, a fragment thereof, an analog thereof, or a derivative thereof.

17. The composition of claim 16, wherein the C3 protein is selected from the group consisting of ETF-3, C3, C3i, C3a, C3b, iC3b, C3c, C3d, C3dg, C3g, C3e and C3f.

18. The composition of claim 16, wherein the C3 protein is present at a concentration of about 0.01 μg/ml to about 1,000 μg/ml.

19. The composition of claim 18, wherein the C3 protein is present at a concentration of about 0.1 μg/ml to about 100 μg/ml.

20. The composition of claim 19, wherein the C3 protein is present at a concentration of about 1 μg/ml to about 10 μg/ml.

21. The composition of claim 16, wherein the C3 protein is present at a physiological concentration that enhances the development of mammalian embryos relative to the development of embryos cultured in a medium without the C3 protein.

22. The composition of claim 16 further comprising a mammalian cell.

23. The composition of claim 22, wherein the mammalian cell is from a preimplantation embryo.

24. The composition of claim 23, wherein the preimplantation embryo has at least 2-4 cells, 4-8 cells, 8-16 cells, 16-32 cells, 32-64 cells, or 64-128 cells.

25. The composition of claim 22, wherein the mammalian cell is a primate cell.

26. The composition of claim 22, wherein the mammalian cell is a non-primate cell selected from the group consisting of canines, felines, mouse, bovines, sheep and pigs.
